SEQUENCE LISTING (6 sequences)

SEQ ID NO: 1 - RNA, 657 nt, Plasmodium vivax
  source 1..657: mol_type = mRNA
atgaactcctactactccctgttcgtgttcttcctggtgcagatcgccctgaagtactccaaggccgccgtgaccgtggacaccatctgcaagaacggccagctggtgcagatgtccaaccacttcaagtgcatgtgcaacgagggcctggtgcacctgtccgagaacacctgcgaggagaagaacgagtgcaagaaggagaccctgggcaaggcctgcggcgagttcggccagtgcatcgagaaccccgaccccgcccaggtgaacatgtacaagtgcggctgcatcgagggctacaccctgaaggaggacacctgcgtgctggacgtgtgccagtacaagaactgcggcgagtccggcgagtgcatcgtggagtacctgtccgagatccagtccgccggctgctcctgcgccatcggcaaggtgcccaaccccgaggacgagaagaagtgcaccaagaccggcgagaccgcctgccagctgaagtgcaacaccgacaacgaggtgtgcaagaacgtggagggcgtgtacaagtgccagtgcatggagggcttcaccttcgacaaggagaagaacgtgtgcctgtcctactccgtgttcaacatcctgaactactccctgttcttcatcatcctgctggtgctgtcctacgtgatc

SEQ ID NO: 2 - RNA, 657 nt, Plasmodium vivax
  source 1..657: mol_type = mRNA
atgaactcctactactccctgttcgtgttcttcctggtgcagatcgccctgaagtactccaaggccgccgtgaccgtggacaccatctgcaagaacggccagctggtgcagatgtccaaccacttcaagtgcatgtgcaacgagggcctggtgcacctgtccgagaacacctgcgaggagaagaacgagtgcaagaaggagaccctgggcaaggcctgcggcgagttcggccagtgcatcgagaaccccgaccccgcccaggtgaacatgtacaagtgcggctgcatcgagggctacaccctgaaggaggacacctgcgtgctggacgtgtgccagtacaagaactgcggcgagtccggcgagtgcatcgtggagtacctgtccgagacccagtccgccggctgctcctgcgccatcggcaaggtgcccaaccccgaggacgagaagaagtgcaccaagaccggcgagaccgcctgccagctgaagtgcaacaccgacaacgaggtgtgcaagaacgtggagggcgtgtacaagtgccagtgcatggagggcttcaccttcgacaaggagaagaacgtgtgcctgtcctactccgtgttcaacatcctgaactactccctgttcttcatcatcctgctggtgctgtcctacgtgatc

SEQ ID NO: 3 - protein, 219 aa, Plasmodium vivax
MNSYYSLFVFFLVQIALKYSKAAVTVDTICKNGQLVQMSNHFKCMCNEGLVHLSENTCEEKNECKKETLGKACGEFGQCIENPDPAQVNMYKCGCIEGYTLKEDTCVLDVCQYKNCGESGECIVEYLSEIQSAGCSCAIGKVPNPEDEKKCTKTGETACQLKCNTDNEVCKNVEGVYKCQCMEGFTFDKEKNVCLSYSVFNILNYSLFFIILLVLSYVI

SEQ ID NO: 4 - protein, 219 aa, Plasmodium vivax
MNSYYSLFVFFLVQIALKYSKAAVTVDTICKNGQLVQMSNHFKCMCNEGLVHLSENTCEEKNECKKETLGKACGEFGQCIENPDPAQVNMYKCGCIEGYTLKEDTCVLDVCQYKNCGESGECIVEYLSETQSAGCSCAIGKVPNPEDEKKCTKTGETACQLKCNTDNEVCKNVEGVYKCQCMEGFTFDKEKNVCLSYSVFNILNYSLFFIILLVLSYVI

SEQ ID NO: 5 - RNA, 75 nt, Homo sapiens
  source 1..75: mol_type = mRNA
atggccatctccggcgtgcccgtgctgggcttcttcatcatcgccgtgctgatgtccgcccaggagtcctgggcc

SEQ ID NO: 6 - RNA, 57 nt, Homo sapiens
  source 1..57: mol_type = mRNA
atgcgcatgcagctgctgctgctgatcgccctgtccctggccctggtgaccaactcc

(19) United States
(12) Patent Application Publication
(10) Pub. No.: US 2026/0364159 A1
(43) Pub. Date: Nov. 19, 2026

(54) A TRANSMISSION-BLOCKING COMPOSITION AGAINST PLASMODIUM VIVAX

(71) Applicant: MAHIDOL UNIVERSITY, Nakhon Pathom (TH)

(72) Inventors: Jetsumon PRACHUMSRI, Bangkok (TH); Wang NGUITRAGOOL, Bangkok (TH); Norbert PARDI, Philadelphia, PA (US); Nawapol KUNKEAW, Bangkok (TH); Sathit PICHYANGKUL, Bangkok (TH)

(73) Assignee: MAHIDOL UNIVERSITY, Nakhon Pathom (TH)

(21) Appl. No.: 19/475,791

(22) PCT Filed: Apr. 20, 2023
(86) PCT No.: PCT/TH2023/000006

§ 371 Date: Oct. 16, 2025

(51) Int. Cl. A61K 39/015; A61K 39/00; A61K 39/39; A61P 33/06

(52) U.S. Cl. CPC A61K 39/015; A61K 39/39; A61P 33/06; A61K 2039/53; A61K 2039/54; A61K 2039/545; A61K 2039/55555; A61K 2039/575; A61K 2039/6018; A61K 2039/6093

(57) ABSTRACT

The present disclosure relates to a composition for prohibiting cross-species infection of malaria caused by P. vivax in a subject. Preferably, the disclosed composition is prepared in the form of a vaccine, which comprises a plurality of polynucleotides each comprising a sequence as setting forth in SEQ ID No. 1 or SEQ ID No. 2, the polynucleotides being expressed in a body of the subject for inducing an immune response reactive against the infection of malaria thereof; a liquid phase of lipid nanoparticles configured to form a protective layer encapsulating the pluralities of polynucleotides within the protective layer; and a pharmaceutically acceptable adjuvant.

### TECHNICAL FIELD

The present disclosure relates to a vaccine against transmission of malaria infection. In more specific, the disclosed vaccine is a mRNA-based vaccine which is effective in blocking transmission or spread of malaria infection between two species, such as transmission between a human host and mosquitos.

### BACKGROUND

Malaria represents the global public health concern. The causative agents of this disease are Plasmodium parasites which are transmitted by female Anopheles mosquitos. Plasmodium falciparum and Plasmodium vivax play the roles as the major parasite species for human malaria. 3.5 billion people are currently at risk of P. vivax infection [1]. Growing number of studies have indicated that P. vivax can potentially cause complications, and death particularly in pregnant women and young children [2]. Particularly, infection with P. vivax is associated with chronic anemia and can lead to maternal anemia, miscarriage, low birth weight and congenital malaria [3]. Compared to Plasmodium falciparum, management of P. vivax infection is more complicated due to the parasite's ability to become dormant in the liver of infected individuals for months to years before reactivation [4]. The reservoir of these dormant parasites, known as hypnozoites, not only extends the clinical attack but also sustains the transmission causing the great epidemiological success around the globe [4]. In addition, P. vivax is more efficient at transmitting to mosquitos [5,6]. Currently, drug applicable for killing hypnozoites are limited to 8-aminoquinolines primaquine and tafenoquine, which are known to result in acute hemolysis in people with glucose-6-phosphate dehydrogenase (G6PD)-deficiency. In addition, primaquine and tafenoquine are contraindicated during pregnancy and breast-feeding. These drugs are not recommended in children less than six months old. Alternative methods to control, cure and/or even to curb spreading of vivax malaria are thus needed [7].

Vaccines are one of the most successful and cost-effective public health tools for eradicating, containing, and reducing infectious diseases. A malaria vaccine that targets sexual or mosquito stage parasites aiming to reduce man-to-mosquito transmission, i.e. a transmission blocking vaccine (TBV), is thus considered an important tool for P. vivax elimination [8]. Because an effective TBV is expected to reduce the overall malaria transmission, it will also have an impact on the intensity of mosquito-to-man transmission as well as the size of hypnozoite reservoir in the population. The Plasmodium vivax Ookinete Surface Protein, Pvs25, a protein antigen expressed on the surface of ookinetes, has been proposed as a leading TBV candidate [9-11] due to its strong transmission blocking efficacy [12, 13]. Study assessing a recombinant Pvs25 formulated with anhydro gel targeting P. vivax malaria transmission revealed that the vaccine significantly reduced the parasite number in mosquitoes but the titer was not sufficient for being an effective vaccine [14]. Another study conducted to test Pvs25/Montanide ISA 51 based transmission blocking vaccine observed a high and functionally active antibody response. Nonetheless, this study encountered the concern of local reactions that ceases further development of the formulation [15]. In view of that, a new and novel vaccination platform is therefore urgently needed, particularly a Pvs25-based transmission blocking vaccine capable of inducing potent and long-lasting transmission blocking immunity yet being safe for clinical application.

Conventional vaccine approaches, such as live attenuated and inactivated pathogens and protein subunit vaccines, employed for development of vaccine against P. vivax in the earlier studies have encountered critical setbacks as mentioned above. Nonetheless, in recent years, various forms of mRNA-based vaccines have proven to be highly effective against cancer and infectious diseases compared to the conventional approaches. One of the most promising vaccine platforms comprises nucleoside-modified mRNA encapsulated in lipid nanoparticles (LNPs). The nucleoside-modified mRNA-LNP vaccines developed by Pfizer/BioNTech and Moderna have demonstrated the effectiveness to curb Covid-19 [17]. A single dose of nucleoside-modified mRNA-LNP vaccine elicited potent and sustained protective neutralizing antibody responses against Zika and influenza viruses in mice and non-human primates [18-20]. Moreover, comparative studies confirmed that the nucleoside-modified mRNA-LNP vaccine outperformed conventional vaccine formats such as MF59-adjuvanted protein subunit and inactivated pathogen vaccines [18]. The nucleoside-modified mRNA-LNP vaccine has the unique ability to potently induce T follicular helper cells that are critical drivers of antibody affinity maturation and the generation of protective neutralizing antibodies [21]. Recently, nucleoside modified mRNA vaccines encoding P. falciparum CSP was found to be immunogenic in mice and protective in homologous and heterologous transgenic rodent models making this a compelling platform for further malaria vaccine development [22], particularly a vaccine effective against P. vivax infection.

### SUMMARY

One object of the present disclosure is to provide a composition capable of eliciting an immune response in a subject against infection of malaria, particularly caused by P. vivax, upon administrating the disclosed composition to the subject through the predetermined route.

Further object of the present disclosure aims to offer a vaccine composition effective in curbing, prohibiting and/or reducing the likelihood of cross-species transmission of malaria which, but not limited to, P. vivax is the causative agent. Particularly, the disclosed composition can induce an immune response in a subject received the vaccine for retarding, prohibiting and/or arresting development of merozoites and/or hypnozoites in the subject and/or mosquitoes fed on the subject through one or more antibodies generated by the subject in association with the vaccine composition received. The subject may not have infected with malaria.

More object of the present disclosure associates to a mRNA-based vaccine composition designed to arrest development of merozoites and/or hypnozoites in a mammalian subject upon administrating one or more pharmaceutically effective dosage of the vaccine composition within a period of time.

Still, another object of the present disclosure is to offer a method for inducing an immune response in a subject by way of administrating a mRNA-based vaccine composition prepared using polynucleotide sequence of Plasmodium vivax ookinete surface protein antigen, namely Pvs25 protein.

At least one of the preceding objects is met, in whole or in part, by the present disclosure, in which one of the embodiments of the present disclosure is a vaccine composition for prohibiting cross-species infection of malaria caused by P. vivax in a subject, or even mosquitoes fed on the subject potentially carrying merozoites and/or hypnozoites of P. vivax. Preferably, the vaccine composition comprises a plurality of polynucleotides each comprising a sequence as setting forth in SEQ ID No. 1 or SEQ ID No. 2, the polynucleotides being expressed in a body of the subject for inducing an immune response reactive against the infection of malaria thereof; a liquid phase of lipid nanoparticles configured to form a protective layer encapsulating the pluralities of polynucleotides within the protective layer; and a pharmaceutically acceptable adjuvant.

In some embodiments, the polynucleotides of the disclosed composition have uridine substituted or replaced by any one or combination of pseudouridine and 1-methyl-pseudouridine.

In more embodiments, the lipid nanoparticles comprise cationic lipid, distearoylphosphatidylcholine (DSPC), cholesterol and PEG. More preferably, the cationic lipid, distearoylphosphatidylcholine (DSPC), cholesterol and PEG are in a molar ratio of 40-60:5-15:30-50:1-5.

For some embodiments, the polynucleotide further comprises a region encoding for at least one signaling peptide, which is incorporated upstream of the sequence of SEQ ID No. 1 or SEQ ID No. 2 in an expression construct bearing the polynucleotide sequence of SEQ ID No. 1 or SEQ ID No. 2. The incorporation of the signaling peptide may facilitate better recognition of the expressed peptide by the host or subject receiving the vaccine in some embodiments. According to several preferred embodiments, the signaling peptide derived from either one of signaling peptide of MHC class II. Some examples of the signaling peptide used are detailed in SEQ ID No. 5 or SEQ ID No. 6.

For more embodiments, the sequence of SEQ ID No. 1 and/or SEQ ID No. 2 is derived from strain Sal I of P. vivax.

Another aspect of the present disclosure involves a method of inducing an immune response in a subject reactive against infection of malaria caused by P. vivax. Essentially, the disclosed method comprises administrating a vaccine composition to the subject by way of intramuscular route or subcutaneous route that the vaccine composition comprises a plurality of polynucleotides each comprising a sequence as setting forth in SEQ ID No. 1 and/or SEQ ID No. 2, the polynucleotides being expressed in a body of the subject for inducing an immune response reactive against the infection of malaria thereof; a liquid phase of lipid nanoparticles configured to form a protective layer encapsulating the pluralities of polynucleotides within the protective layer; and a pharmaceutically acceptable adjuvant. Preferably, the polynucleotides have uridine substituted by any one or combination of pseudouridine and 1-methyl-pseudouridine.

According to several embodiments of the disclosed method, the lipid nanoparticles comprise cationic lipid, distearoylphosphatidylcholine (DSPC), cholesterol and PEG. Preferably, the cationic lipid, distearoylphosphatidylcholine (DSPC), cholesterol and PEG are in a molar ratio of 40-60:5-15:30-50:1-5.

### BRIEF DESCRIPTION OF THE DRAWINGS

FIG. 1 is a gel picture showing various constructs with different modifications as explained in Example 1 were successfully expressed for investigation through subsequent experiments;

FIG. 2 are graphs showing results about antigen specific antibody response in mice immunized with nucleoside-modified constructs incorporated with the polynucleotides sequence of Pvs25;

FIG. 3 are micrographs showing the results obtained from testing the pooled plasma of the immunized mice against native antigen on the surface of P. vivax ookinetes that the uppermost panels show nuclear staining with DAPI followed by the second row showing staining with anti-mouse IgG Alexa Fluor 488 secondary antibody, the third row presenting the merged images, and the bottom row showing the differential Interference Contrast (DIC) images (points represents individual mice and horizontal lines denote geometric mean with 95% CI as well as vertical lines represent error bar)

FIG. 4 is a graph showing the functional activity of antisera induced by Pvs25 mRNA-LNP vaccines with TRA represents the % reduction in the mean oocyst density in the presence of each specified immune-serum relative to the non-immune serum (using One-way ANOVA with Bonferroni correction, *p<0.05);

FIG. 5 is a graph showing the Pvs25-specific antibody response in the mice using ELISA (n=17 per group) at 1 month after the prime vaccination (Month 0) and 1 month after boost vaccination (Month 1);

FIG. 6 includes graphs showing (a) activity indices of the anti-Pvs25 and (b) IgG2a/IgG1 ratio of the anti-Pvs25 (using One-way ANOVA with Bonferroni correction, *p<0.05);

FIG. 7 includes graphs showing the frequency of production of different interleukins and interferons by (a) CD4-T cells and (b) CD8-T cells of the mice immunized using different constructs prepared;

FIG. 8 is a graph showing the immune response induced in mice immunized with different constructs prepared over a period of 7 months; and

FIG. 9 includes graphs showing transmission reducing activity (TRA) induced by different immunization regimens at the first month and the seventh month;

FIG. 10 is a graph showing relationship between TRA (from 9) and total IgG produced; and

FIG. 11 is a listing showing SEQ ID No. 1 and SEQ ID No. 2, SEQ ID No. 3, SEQ ID No. 4, SEQ ID No. 5, and SEQ ID No. 6.

### DETAILED DESCRIPTION

Hereinafter, the disclosure shall be described according to the preferred embodiments and by referring to the accompanying description and drawings. However, it is to be understood that referring the description to the preferred embodiments of the disclosure and to the drawings is merely to facilitate discussion of the various disclosed embodiments and it is envisioned that those skilled in the art may devise various modifications without departing from the scope of the appended claim.

The term “polynucleotide” or “nucleic acid” as used herein designates mRNA, RNA, CRNA, cDNA or DNA. The term typically refers to oligonucleotides greater than 30 nucleotide residues in length.

The term “gene” as used herein may refer to a DNA sequence with functional significance. It can be a native nucleic acid sequence, or a recombinant nucleic acid sequence derived from natural source or synthetic construct. The term “gene” may also be used to refer to, for example and without limitation, a cDNA and/or an mRNA encoded by or derived from, directly or indirectly, genomic DNA sequence.

According to one aspect of the present disclosure, a composition for prohibiting cross-species infection of malaria caused by P. vivax in a subject is provided. Preferably, the composition is configured to inhibit, arrest, prohibit and/or cease development of merozoites and/or hypnozoites potentially carried by a human subject and/or a transmitting agent such as female mosquitoes of the genus Anopheles. The disclosed vaccine composition generally comprises a plurality of polynucleotides each comprising a sequence as setting forth in SEQ ID No. 1 and/or SEQ ID No. 2, the polynucleotides being expressed in a body of the subject for inducing an immune response reactive against the infection of malaria thereof; a liquid phase of lipid nanoparticles configured to form a protective layer encapsulating the pluralities of polynucleotides within the protective layer; and a pharmaceutically acceptable adjuvant.

Referring to sequence listing of SEQ ID No. 1, the inventors of the present disclosure employ a polynucleotide sequence of Plasmodium vivax ookinete surface protein antigen, namely Pvs25, protein as the template or base template for preparing the polynucleotide sequence SEQ ID No. 1 in the form of mRNA. With the utilization of mRNA instead of conventional protein-based vaccine, the disclosed composition is relatively safer to use considering that mRNA is a non-infectious and non-integrating platform without exposing the subject to the risk of infection or insertional mutagenesis. Moreover, mRNA is the minimal genetic vector; therefore, anti-vector immunity can be avoided, and mRNA vaccines can be administered repeatedly. Additionally, the mRNA platform of the present disclosure is degradable by normal cellular processes, and it in vivo half-life can be regulated through various modifications introduced and delivery methods used to further reduce the likelihood of the occurrence of any undesired outcome. It is important to note that the polynucleotide used in the present disclosure for preparing the vaccine composition can be of natural, preferably further processed by one or more purification steps, or synthetic origin. The Pvs25 sequence used can be with or without modification depending on the designated outcome to be attained as further described in the following. For instance, in some embodiments, the polynucleotide has uridine, which is found in natural or general mRNA template, substituted by any one or combination of pseudouridine and 1-methyl-pseudouridine. The employment of pseudouridine and/or 1-methyl-pseudouridine incorporated in the disclosed composition ensures higher level of protein expression in the subject. The substitution of uridine by pseudouridine and/or 1-methyl-pseudouridine also lowers the risk of serious inflammatory responses in the subject after receiving the disclosed vaccine composition.

Pursuant to more embodiments of the disclosed composition, the polynucleotide used may comprise a I130T mutation as an additional modification made towards the polynucleotide sequence used to improve overall performance of the disclosed composition. The sequence of the mentioned polynucleotide having the intended mutation is disclosed in SEQ ID No. 2 of FIG. 11. Preferably, the included mutation enhances the transmission blocking activity towards other variants of Plasmodium parasites, particularly a variant of Plasmodium vivax carrying the aforesaid mutation. Further, the sequences of the peptides correspondingly encoded by the SEQ ID No. 1 and SEQ ID No. 2 are shown in SEQ ID No. 3 and SEQ ID No. 4.

For some embodiments, the polynucleotide further comprises a region encoding for at least one signaling peptide, which is incorporated upstream of the sequence of SEQ ID No. 1 or SEQ ID No. 2 in an expression construct bearing the polynucleotide sequence of SEQ ID No. 1 or SEQ ID No. 2. The incorporation of the signaling peptide may facilitate better recognition of the expressed peptide by the host or subject receiving the vaccine in some embodiments. According to several preferred embodiments, the signaling peptide derived from either one of signaling peptide of MHC class II. Some examples of the signaling peptide used are detailed in SEQ ID No. 5 or SEQ ID No. 6. Particularly, in some embodiments, each of the plurality of polynucleotides further comprises SEQ ID No. 5 and/or SEQ ID No. 6 located upstream or downstream of SEQ ID No. 1 or SEQ ID No. 2.

As described in the setting forth, a liquid phase composed of LNPs are used to form a protective layer to encapsulate, enclose, or wrap the plurality. By mixing the LNP along with the polynucleotides under a regulated and controlled condition, the LNPs self-assemble into 80-100 nm particles. The protective layer established though the LNPs creates a neutral surface in relation to the cellular environment allowing the encapsulated polynucleotides to shunt extensive binding to serum proteins prior to expression. More specifically, the mRNA-loaded LNPs are taken up via endocytosis then go through the endosomal pathway, get disrupted by endosomal acidification, and a fraction of the RNA escapes from the endosomes to enter the cytosol, where protein production from mRNA occurs [35-37]. In some embodiments, the lipid nanoparticles comprise cationic lipid, distearoylphosphatidylcholine (DSPC), cholesterol and PEG. More preferably, the cationic lipid, distearoylphosphatidylcholine (DSPC), cholesterol and PEG are in a molar ratio of 40-60:5-15:30-50:1-5.

For several embodiments, the adjuvant can be any one or combination of an aqueous solution, a saline solution, Ringer's solution, isotonic sodium chloride, synthetic monoglycerides, synthetic diglycerides, polyethylene glycols, glycerin, propylene glycol, antibacterial agents, antioxidants, chelating agents, buffering agents, tonicity modifying agents, and cryoprotectants. Preferably, the antibacterial agents can be benzyl alcohol, methyl paraben, and the like. Preferably, the antioxidants can be ascorbic acid, sodium bisulfite, and the like. Preferably, the buffering agents can be acetates, citrates or phosphates, and the like. Preferably, the tonicity modifying agents can be sodium chloride, dextrose, and the like. Preferably, the cryoprotectants can be sucrose, trehalose, and the like. Also, the chelating agents such as ethylenediaminetetraacetic acid can be used as well.

Another aspect of the present disclosure relates to a method of inducing an immune response in a subject reactive against infection of malaria caused by P. vivax. The immune response induced shall result in production of an antibody reactive against Pvs25 antigen which in turn arrests or inhibits development of P. vivax, particularly merozoites and/or hypnozoites, in the subject. It is crucial to note that the antibodies produced can be transferred to malaria transmitting agents, such as female mosquitoes of the genus Anopheles, happened to feed on the blood of the subject carrying the antibodies. These antibodies are configured to attain the same outcome in the mosquitoes to arrest or inhibit development of the P. vivax merozoites and/or hypnozoites. Thus, the mosquitoes bearing these antibodies shall fail to effectuate transmission of malaria subsequently shrinking the pool of malaria infectious agents available. Through the inhibition mechanism, the disclosed method is able to reduce or prevent the cross-species transmission of malaria particularly caused by P. vivax. The disclosed method preferably comprises the steps of administrating a vaccine composition to the subject by way of intramuscular route or subcutaneous route. More specifically, the vaccine composition used in the disclosed method comprises a plurality of polynucleotides each comprising a sequence as setting forth in SEQ ID No. 1, the polynucleotides being configured to express in a body of the subject for inducing an immune response reactive against the infection of malaria thereof; a liquid phase of lipid nanoparticles configured to form a protective layer encapsulating the pluralities of polynucleotides within the protective layer; and a pharmaceutically acceptable adjuvant.

Corresponding to the vaccine composition mentioned above, the polynucleotides of the vaccine composition used in the disclosed method has uridine substituted by any one or combination of pseudouridine and 1-methyl-pseudouridine to improve expression of the antigenic protein in the subject.

Likewise, the lipid nanoparticles found in the vaccine composition used for the disclosed method comprises cationic lipid, distearoylphosphatidylcholine (DSPC), cholesterol and PEG. More preferably, the cationic lipid, distearoylphosphatidylcholine (DSPC), cholesterol and PEG are in a molar ratio of 40-60:5-15:30-50:1-5.

The following example is intended to further illustrate the disclosure, without any intent for the disclosure to be limited to the specific embodiments described therein.

### Example 1

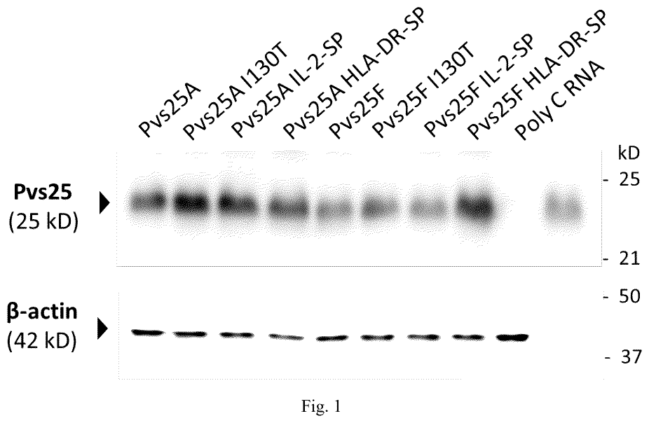

Western blotting was performed with Pvs25-specific antibodies confirming the expression of Pvs25 from all mRNA-LNPs in human monocyte-derived dendritic cells (mo-Dcs). The results are present in FIG. 1. All eight constructs were designed based on the sequence of the Pvs25 gene from the reference P. vivax strain Sal I. Four constructs express Pvs25 without the C-terminal GPI anchor. Two of them (Pvs25A and Pvs25A I130T) have the wild type signal peptide sequence; one of which (Pvs25A I130T) contains the I130T substitution predominant in the Asian P. vivax isolates. The other two (Pvs25A IL-2 SP and Pvs25A HLA-DR SP) have exogenous signal peptide sequences (human IL-2 and HLA-DR) which are known to enhance protein expression. The other four constructs encode Pvs25 with the C terminal GPI anchor. Pvs25F encodes the full-length sequence of the Pvs25 gene from Sal I. The remaining three constructs (Pvs25F I130T, Pvs25F IL-2 SP, and Pvs25F HLA-DR SP) contain the full-length sequence of Pvs25 with a) the 1130T mutation, b) the IL-2 signal peptide, or c) the HLA-DR signal peptide. Cells transfected with polycytidine (PolyC) RNA-LNP was used as the negative control. Recombinant protein (Pvs25A) produced by the wheat germ cell free system was used as the positive control.

### Example 2

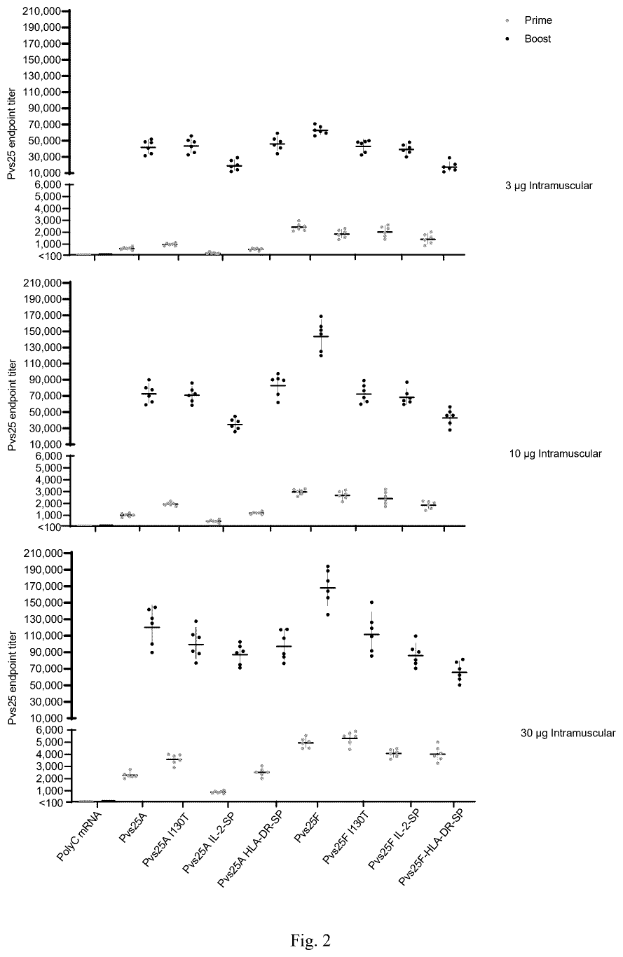

Experiments was performed to assess immunity response after administrating the various constructs prepared in example 1 to mice. The mice were immunized using the various Pvs25 mRNA-LNPs constructs prepared. Particularly, BALB/C mice were immunized 3 μg, 10 μg or 30 μg of Pvs25 mRNA-LNPs via intramuscular injection in a 4 week interval prime: boost regimen (N=6 per group). In the control group, mice received 30 μg of PolyC RNA-LNP (N=6). The results are present in FIG. 2.

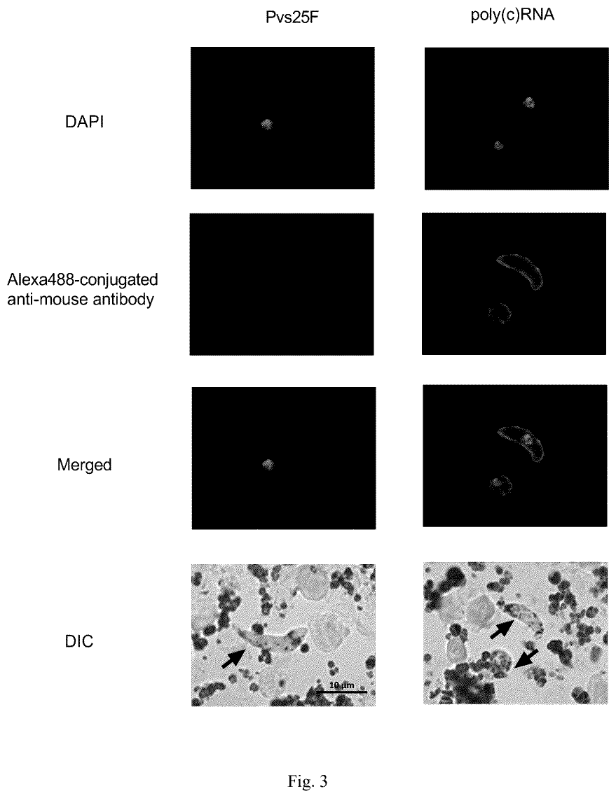

Further, pooled plasma from the immunized BALB/c mice, after the final immunization with 30 μg of Pvs25F mRNA-LNP (Pvs25F) or Poly C mRNA-LNP (PolyC), was used to assess reactivity towards the native antigen on the surface of P. vivax ookinetes by immunofluorescence assay (IFA). The results are shown in FIG. 3.

### Example 3

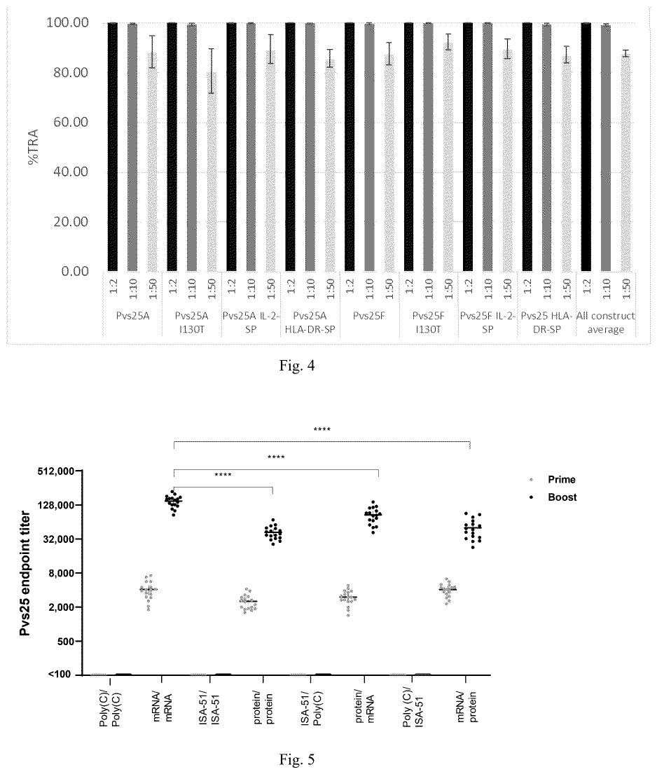

The direct membrane feeding assay (DMFA) was performed to evaluate transmission reducing activity (TRA) of immunized mouse antisera at different dilutions. Lab-reared uninfected mosquitoes were fed on P. vivax malaria patient blood (n=5) suspended with immune (from mice that received 30 μg of each of the eight Pvs25 mRNA-LNPs) or non-immune (from mice received 30 μg of poly C RNA-LNP sera, then the mosquitoes were dissected 7 days post feeding and oocysts were counted under the light microscope to derive the results thereof as shown in FIG. 4.

### Example 4

The present disclosure compared the immunogenicity and kinetics of the immune response of four different immunization regimens: a) Pvs25F mRNA-LNP homologous prime-boost vaccination b) Pvs25 protein (with ISA-51 adjuvant) homologous prime-boost vaccination, c) Pvs25F mRNA-LNP/Pvs25 protein heterologous vaccination and d) Pvs25 protein/Pvs25F mRNA-LNP heterologous vaccination. Mice were randomly assigned into 8 groups (n=17 mice per group). In 4 vaccination groups, mice were vaccinated with homologous and heterologous prime-boost regimens (10 μg mRNA or 10 μg recombinant protein vaccine, 4 weeks between the prime and boost). In the other 4 groups, 10 μg Poly(C) RNA-LNP or Montanide ISA-51 VG was administered as negative controls for each vaccination group.

Sera from the control groups had no detectable Pvs25-specific antibody response by ELISA (FIG. 5). The mRNA/mRNA homologous prime-boost vaccination generated the strongest Pvs25-specific antibody response with GMT of ~140,000. The protein/protein homologous vaccination had GMT of ~40,000. The protein/mRNA vaccination had GMT of ~80,000 and the mRNA/protein vaccination yielded GMT of ~46,000.

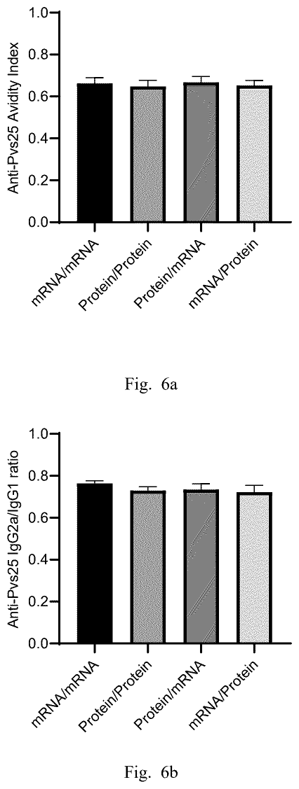

The present disclosure further characterized the quality of Pvs25-specific antibodies. IgG1 and IgG2a subclasses expressed as the IgG2a/IgG1 ratio and antigen-antibody avidity index. Here, pooled serum was used to determine the IgG2a/IgG1 ratio and the avidity index (FIGS. 6a and 6b). The IgG subclass patterns of all vaccination regimens were similar with the IgG2a/IgG1 ratio of ~0.75. The avidity indices were also similar across the groups.

### Example 5

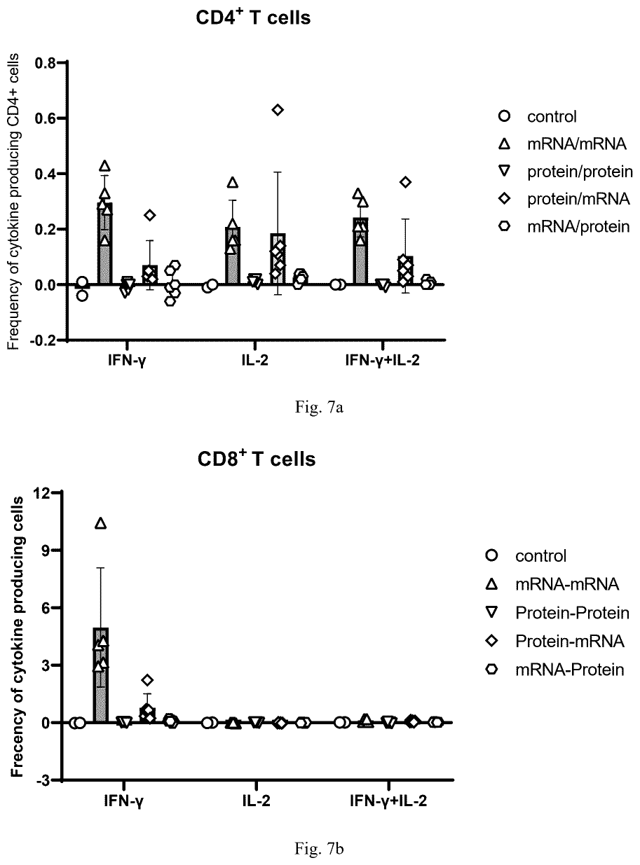

Experiment was performed to assess the production of IFN-γ and IL-2 by CD4+ and CD8+ T cells. Particularly, splenocytes of the immunized mice were obtained at Month 1 after the immunization then subjecting the splenocytes to Pvs25 peptide stimulation to analyze the IFN-γ and IL-2 produced by CD4+ and CD8+ T cells with controls (n=2) selected from the 4 controls predetermined. The results were summarized respectively in FIGS. 7a and 7b.

More specifically, mouse splenocytes from the homologous and heterologous prime-boost experiment were evaluated for cellular immune responses. The mRNA/mRNA homologous prime-boost vaccination induced the most robust Pvs25-specific CD4+ and CD8+ T cell responses as measured by IFN-γ and IL-2 production, while the protein/protein vaccination barely induced T cells. The results were summarized respectively in FIGS. 7a and 7b. Likewise, the mRNA/mRNA homologous vaccination elicited the strongest memory B cell response whereas this was almost absent in the protein/protein homologous vaccination. The protein/mRNA heterologous vaccination gave positive but intermediate cellular responses while the mRNA/protein vaccination elicited very low cellular response similar to the protein/protein vaccination. The result is presented in FIG. 7.

### Example 6

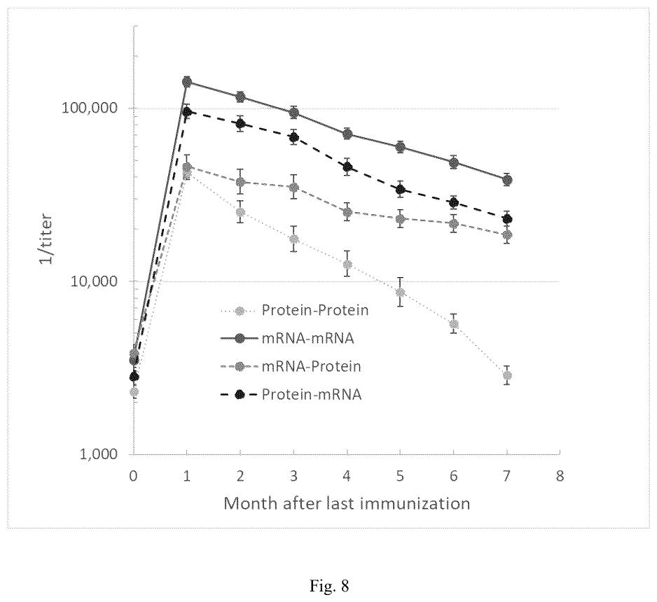

The present disclosure also used ELISA to determine Pvs25 antibodies response in the mice immunized by the four different constructs prepared over a period of 7 months with measurements being conducted at each of the month. Nine mice per group were maintained for 7 months after the booster dose at Month 0. The results are summarized in FIG. 8. Particularly, Pvs25 antibody levels were followed monthly for 7 months post boost vaccination to assess the durability of antibody responses (FIG. 8). The antibody levels peaked at 1 month post boost in all vaccination groups and declined over the subsequent months. In agreement with the previous results, GMTs were highest in the mRNA/mRNA group and the lowest in the protein/protein group. By the 7th month, the antibody level had a GMT of 36,000 for mRNA/mRNA, 2,300 for protein/protein, 25,000 for protein/mRNA, and 18,000 for mRNA/protein.

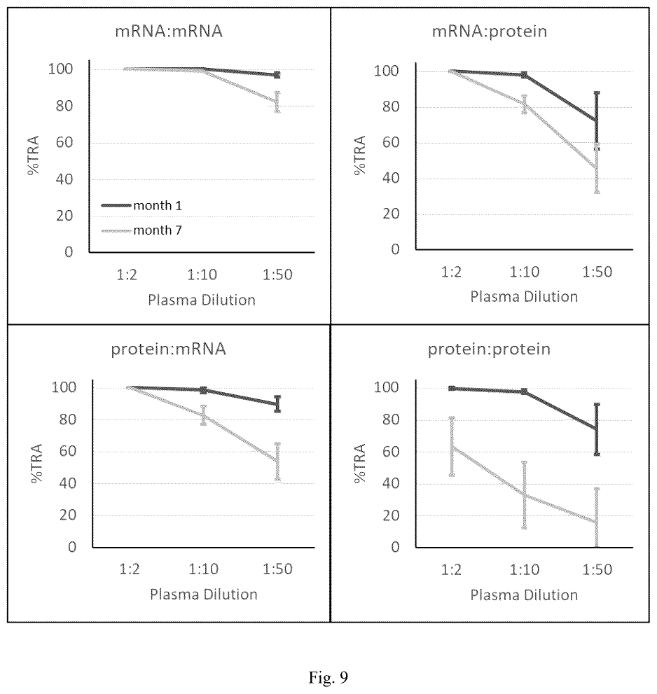

Further, investigation was done towards transmission reducing activity of the immunized mice. Particularly, pooled sera obtained from the mice at the 1st month and 7th month were subjected to direct membrane feeding assay (DMFA) against P. vivax-infected blood from four different patients. Transmission reducing activity (TRA) was determined at 1:2, 1:10, and 1:50 serum dilutions with the obtained results shown in FIG. 9. Particularly, after the booster immunization of the homologous and heterologous prime-boost experiment, the Pvs25 antibody levels in mice (8-9 per group) were followed monthly for 7 months to assess the durability of the antibody response by ELISA. The result is presented in FIG. 9. A similar pattern was observed when the TRA was followed over time; the TRA was the highest one month after the boost (FIG. 9).

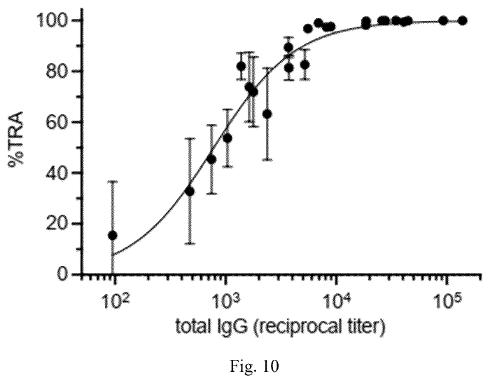

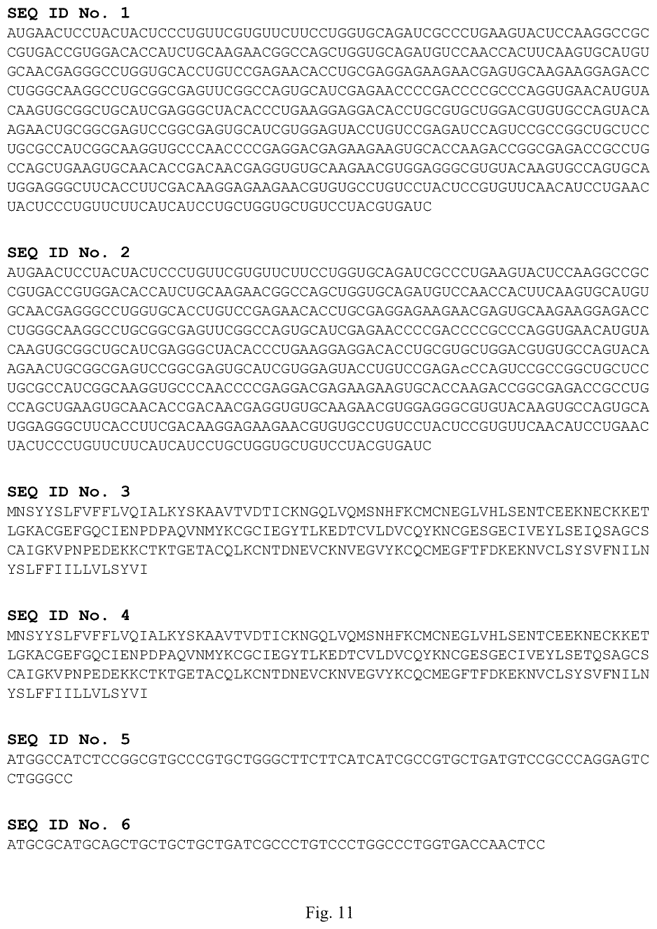

At this time, all vaccination regimens rendered the full efficacy (100% TRA) at serum dilution 1:2. However, for the same 1:2 dilution the TRA of protein/protein vaccination declined to 63% whereas the other three vaccination strategies retained high efficacy >99% at 7 months post boost. Among the 4 vaccination regimens, the mRNA/mRNA group exhibited the most durable functional response, as the TRA remained over 80% even at 1:50 dilution in this group. When the TRA data across all immunization regimens are pooled, there is a clear dose response relationship between TRA and total IgG as expected (FIG. 10). The half-maximal inhibition concentration (IC50) of Pvs25 total IgG was 781 (CI95: 564-1003) reciprocal titer unit.

It is to be understood that the present disclosure may be embodied in other specific forms and is not limited to the sole embodiment described above. However, modification and equivalents of the disclosed concepts such as those which readily occur to one skilled in the art are intended to be included within the scope of the claims which are appended thereto.

### REFERENCES

1. WHO. World malaria report 2019. 2019.
2. Naing C, Whittaker M A, Nyunt Wai V, Mak J W. Is Plasmodium vivax malaria a severe malaria?: a systematic review and meta-analysis. PLOS Negl Trop Dis. 2014; 8 (8):e3071.
3. Anstey N M, Douglas N M, Poespoprodjo J R, Price R N. Plasmodium vivax: clinical spectrum, risk factors and pathogenesis. Adv Parasitol. 2012; 80:151-201.
4. Puji B S Asih DSaJKB. Challenges in the Control and Elimination of Plasmodium vivax Malaria. In: Dev SMaV, editor. Towards Malaria Elimination-A Leap Forward: IntechOpen; 2018.
5. Roth A, Maher S P, Conway A J, Ubalee R, Chaumeau V, Andolina C, et al. A comprehensive model for assessment of liver stage therapies targeting Plasmodium vivax and Plasmodium falciparum. Nat Commun. 2018; 9 (1): 1837.
6. Bennett J W, Yadava A, Tosh D, Sattabongkot J, Komisar J, Ware L A, et al. Phase 1/2a Trial of Plasmodium vivax Malaria Vaccine Candidate VMP001/AS01B in Malaria-Naive Adults: Safety, Immunogenicity, and Efficacy. PLOS Negl Trop Dis. 2016; 10 (2):e0004423.
7. Watson J, Taylor W R J, Bancone G, Chu C S, Jittamala P, White N J. Implications of current therapeutic restrictions for primaquine and tafenoquine in the radical cure of vivax malaria. PLOS Negl Trop Dis. 2018; 12 (4):e0006440.
8. Roth A, Maher S P, Conway A J, Ubalee R, Chaumeau V, Andolina C, et al. A comprehensive model for assessment of liver stage therapies targeting Plasmodium vivax and Plasmodium falciparum. Nat Commun. 2018; 9 (1): 1837.
9 Arakawa, T. et al. Tricomponent complex loaded with a mosquito-stage antigen of the malaria parasite induces potent transmission-blocking immunity. Clin Vaccine Immunol 21, 561-569, doi: 10.1128/CVI.00053-14 (2014).
10 Blagborough, A. M. et al. Transmission blocking potency and immunogenicity of a plant-produced Pvs25-based subunit vaccine against Plasmodium vivax. Vaccine 34, 3252-3259, doi: 10.1016/j.vaccine.2016.05.007 (2016).
11 Mizutani, M. et al. Baculovirus-vectored multistage Plasmodium vivax vaccine induces both protective and transmission-blocking immunities against transgenic rodent malaria parasites. Infect Immun 82, 4348-4357, doi: 10.1128/IAI.02040-14 (2014).
12 Hisaeda, H. et al. Antibodies to malaria vaccine candidates Pvs25 and Pvs28 completely block the ability of Plasmodium vivax to infect mosquitoes. Infect Immun 68, 6618-6623 (2000).
13 Sattabongkot, J. et al. Blocking of transmission to mosquitoes by antibody to Plasmodium vivax malaria vaccine candidates Pvs25 and Pvs28 despite antigenic polymorphism in field isolates. Am J Trop Med Hyg 69, 536-541 (2003)
14 Markin, E. M. et al. Phase 1 vaccine trial of Pvs25H: a transmission blocking vaccine for Plasmodium vivax malaria. Vaccine. 2005 May 2; 23 (24): 3131-3138. doi: 10.1016/j.vaccine.2004.12.019
15 Wu, Y. et al. “Phase 1 trial of malaria transmission blocking vaccine candidates Pfs25 and Pvs25 formulated with montanide ISA 51” PloS one vol. 3,7 e2636. 9 Jul. 2008, doi: 10.1371/journal.pone.0002636
16. Pardi N, Hogan M J, Porter F W, Weissman D. mRNA vaccines—a new era in vaccinology. Nat Rev Drug Discov. 2018; 17 (4): 261-79.
17. Chaudhary N, Weissman D, Whitehead K A. mRNA vaccines for infectious diseases: principles, delivery and clinical translation [published online ahead of print, 2021 Aug. 25] [published correction appears in Nat Rev Drug Discov. 2021 Sep. 21]. Nat Rev Drug Discov. 2021; 1-22.
18. Pardi N, Hogan M J, Naradikian M S, Parkhouse K, Cain D W, Jones L, et al. Nucleoside-modified mRNA vaccines induce potent T follicular helper and germinal center B cell responses. J Exp Med. 2018; 215 (6): 1571-88.
19. Pardi N, Hogan M J, Pelc R S, Muramatsu H, Andersen H, DeMaso C R, et al. Zika virus protection by a single low-dose nucleoside-modified mRNA vaccination. Nature. 2017; 543 (7644): 248-51.
20. Pardi N, Parkhouse K, Kirkpatrick E, McMahon M, Zost S J, Mui B L, et al. Nucleoside-modified mRNA immunization elicits influenza virus hemagglutinin stalk-specific antibodies. Nat Commun. 2018; 9 (1): 3361.
21. Crotty S. T Follicular Helper Cell Biology: A Decade of Discovery and Diseases. Immunity. 2019; 50 (5): 1132-48
22. Mallory K L, Taylor J A, Zou X, et al. Messenger RNA expressing PfCSP induces functional, protective immune responses against malaria in mice. NPJ Vaccines. 2021; 6 (1): 84.

1. A vaccine composition for prohibiting cross-species infection of malaria caused by Plasmodium vivax in a subject comprising:
a plurality of polynucleotides each comprising a sequence as setting forth in SEQ ID No. 1 or SEQ ID No. 2, the polynucleotides being expressed in a body of the subject for inducing an immune response reactive against the infection of malaria thereof;
a liquid phase of lipid nanoparticles configured to form a protective layer encapsulating the pluralities of polynucleotides within the protective layer; and
a pharmaceutically acceptable adjuvant.

2. The vaccine composition of claim 1, wherein the polynucleotides have uridine substituted by any one or combination of pseudouridine and 1-methyl-pseudouridine.

3. The vaccine composition of claim 1, wherein the lipid nanoparticles comprise cationic lipid, distearoylphosphatidylcholine (DSPC), cholesterol and PEG.

4. The vaccine composition of claim 3, wherein the cationic lipid, distearoylphosphatidylcholine (DSPC), cholesterol and PEG are in a molar ratio of 40-60:5-15:30-50:1-5.

5. The vaccine composition of claim 1, wherein the sequence of SEQ ID No. 1 and/or SEQ ID No. 2 is derived from strain Sal I of P. vivax.

6. The vaccine composition of claim 1, wherein each of the plurality of polynucleotides further comprises SEQ ID No. 5 and/or SEQ ID No. 6 located upstream or downstream of SEQ ID No. 1 or SEQ ID No. 2.

7. The vaccine composition of claim 1, wherein the pharmaceutically acceptable adjuvant is any one or combination of an aqueous solution, a saline solution, Ringer's solution, isotonic sodium chloride, synthetic monoglycerides, synthetic diglycerides, polyethylene glycols, glycerin, propylene glycol, antibacterial agents, antioxidants, chelating agents, buffering agents, tonicity modifying agents, and cryoprotectants.

8. A method of inducing an immune response in a subject reactive against infection of malaria caused by P. vivax comprising:
administrating a vaccine composition to the subject by way of intramuscular route or subcutaneous route, the vaccine composition comprising
a plurality of polynucleotides each comprising a sequence as setting forth in SEQ ID No. 1 or SEQ ID No. 2, the polynucleotides being expressed in a body of the subject for inducing an immune response reactive against the infection of malaria thereof;
a liquid phase of lipid nanoparticles configured to form a protective layer encapsulating the pluralities of polynucleotides within the protective layer; and
a pharmaceutically acceptable adjuvant.

9. The method of claim 8, wherein the polynucleotides have uridine substituted by any one or combination of pseudouridine and 1-methyl-pseudouridine.

10. The method of claim 8, wherein the lipid nanoparticles comprise cationic lipid, distearoylphosphatidylcholine (DSPC), cholesterol and PEG.

11. The method of claim 8, wherein the wherein the cationic lipid, distearoylphosphatidylcholine (DSPC), cholesterol and PEG are in a molar ratio of 40-60:5-15:30-50:1-5.
